(19) United States
(12) Patent Application Publication
(10) Pub. No.: US 2007/0037854 A1
(43) Pub. Date: Feb. 15, 2007

(54) Process for preparing sulfonamide-containing indole compounds

(76) Inventors: Kenji Hayashi, Ibaraki (JP); Taichi Abe, Ibaraki (JP); Naoki Ozeki, Ibaraki (JP); Hiroshi Akamatsu, Ibaraki (JP)

Correspondence Address: BIRCH STEWART KOLASCH & BIRCH, PO BOX 747, FALLS CHURCH, VA, 22040-0747, US

(21) Appl. No.: 10/571,285

(22) PCT Filed: Sep. 1, 2004
(86) PCT No.: PCT/JP04/12650

§ 371 Date: Mar. 9, 2006

(30) Foreign Application Priority Data
Sep. 10, 2003 (JP) 2003-318974

(51) Int. Cl. A61K 31/4439; A61K 31/404; C07D 403/02; C07D 209/18

(52) U.S. Cl. 514/337; 514/415; 546/277.4; 548/495

(57) ABSTRACT

A process for preparing a compound (5a) represented by the following formula: 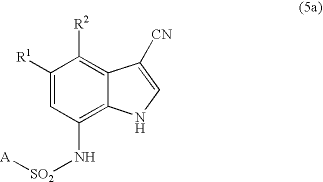
wherein R¹ and R² each independently represent hydrogen, C₁₋₄ alkyl, etc. and A represents cyanophenyl, etc., characterized by reacting a compound (3a) represented by the following formula: 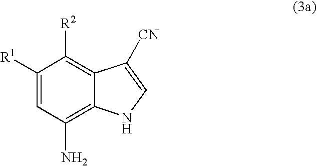
wherein R¹ and R² have the same definitions as above, with a compound represented by the formula A—SO₂Cl, wherein A has the same definition as above, in the presence of a base, in a mixed solvent of water and C₁₋₆ alkyl acetate.

### TECHNICAL FIELD

The present invention relates to a process for preparing sulfonamide-containing indole compounds which are useful as antitumor agents with angiogenesis-inhibitory action.

### BACKGROUND ART

Sulfonamide-containing indole compounds useful as antitumor agents with angiogenesis-inhibitory action are reported in Patent document 1, which discloses sulfonamide-containing indole compounds such as N-(3-cyano-4-methyl-1H-indol-7-yl)-3-cyanobenzenesulfonamide, and a process for preparing the same.

[Patent document 1] WO00/50395

### DISCLOSURE OF THE INVENTION

Problems to be Solved by the Invention

The followings may be mentioned as features of the process for preparing sulfonamide-containing indole compounds described in the aforestated document.
(1) Cyanation reaction is carried out after isolating a formylation reaction product, the two reactions (formylation and cyanation) are conducted in separate steps, and there is a possible obstacle to improve a yield.
(2) Tetrahydrofuran is used as the solvent for reaction of an aminoindole derivative with a sulfonyl chloride derivative, and tetrahydrofuran is not suitable for concentration because it tends to produce peroxides.
(3) Because large amounts of an organic solvent and water must be added in the extraction step after the reaction, precipitation of the product is a problem during the extraction step.

Considering these issues, the process for preparing sulfonamide-containing indole compounds described in the aforestated document is not satisfactory as an industrial preparing process. It is therefore an object of the present invention to overcome the aforementioned problems by providing a useful process for preparing sulfonamide-containing indole compounds.

Means for Solving the Problems

As a result of much avid research in light of the circumstances described above, the present inventors have discovered that the production steps can be shortened and stabilized by:
(1) carrying out the two reactions of formylation and cyanation in one pot, and
(2) changing the reaction solvent/extraction solvent for the aminoindole derivative and sulfonyl chloride derivative, and have thereupon completed this invention.

Specifically, the present invention provides the following [1] to [3].

[1] A process for preparing a compound (5a) represented by the following formula: 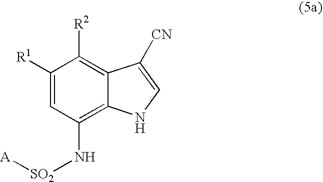
wherein R¹ and R² each independently represent hydrogen, C₁₋₄ alkyl or halogen, and A represents cyanophenyl, aminosulfonylphenyl, aminopyridyl, aminopyrimidyl, halogenopyridyl or cyanothiophenyl, characterized by reacting a compound (3a) represented by the following formula: 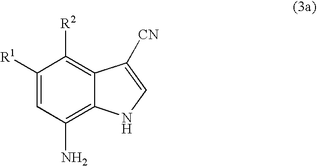
wherein R¹ and R² have the same definitions as above, with a compound represented by the formula A—SO₂Cl, wherein A has the same definition as above, in the presence of a base, in a mixed solvent of water and C₁₋₆ alkyl acetate.

[2] A process for preparing a compound (5a) represented by the following formula: 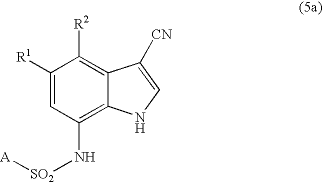
wherein R¹ and R² each independently represent hydrogen, C₁₋₄ alkyl or halogen, and A represents cyanophenyl, aminosulfonylphenyl, aminopyridyl, aminopyrimidyl, halogenopyridyl or cyanothiophenyl, characterized by reacting a compound (1 a) represented by the following formula: 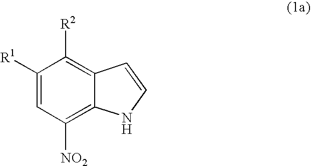
wherein R¹ and R² have the same definitions as above, with a phosphorus oxyhalide or thionyl chloride in dimethylformamide, then adding hydroxylamine hydrochloride to the reaction mixture to allow reaction therewith to afford a compound (2a) represented by the following formula: 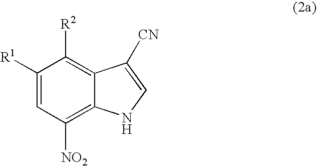
wherein R¹ and R² have the same definitions as above, and then subjecting the compound (2a) to reduction reaction to afford a compound (3a) represented by the following formula: 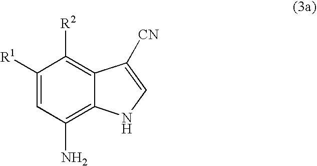
wherein R¹ and R² have the same definitions as above, and reacting the compound (3a) with a compound represented by the formula A—SO₂Cl, wherein A has the same definition as above, in the presence of a base, in a mixed solvent of water and C₁₋₆ alkyl acetate.

[3] A process according to [1] or [2], wherein R² is methyl, R¹ is hydrogen and A is 3-cyanophenyl.

### EFFECT OF THE INVENTION

By carrying out subsequent cyanation without reaction treatment (such as isolation of the formylated compound) after formylation, the reaction is shortened by one step and the yield is improved.

Moreover, the following merits are achieved by changing the reaction solvent from tetrahydrofuran, which is dangerous when concentrated, to a mixed solvent of water and C₁₋₆ alkyl acetate. (1) Safety of the concentration procedure is assured, (2) precipitation of the product is avoided, and (3) the total amount during extraction can be reduced since the reaction solvent also serves as the extraction solvent.

In other words, it is possible to provide a more efficient process for preparing sulfonamide-containing indole compounds which are useful as antitumor agents with angiogenesis-inhibitory action.

### BEST MODE FOR CARRYING OUT THE INVENTION

The present invention will now be explained in greater detail.

Throughout the present specification, the structural formulas for compounds may show only one isomer form for convenience, but the present invention encompasses all isomers implied by the structures of the compounds of the invention, including geometric isomers, optical isomers based on asymmetric carbons, stereoisomers and tautomers, and mixtures of isomers, and is not limited merely to the formulas shown for convenience.

The compounds may also form salts, and all of their anhydrates, hydrates and solvates are also encompassed within the scope of the invention. Unless otherwise specified, the compounds may be amorphous or crystalline, with no particular restrictions on the crystalline form.

The term “halogen” as used throughout the present specification refers to fluorine, chlorine, bromine and iodine.

The term “C₁₋₄ alkyl” as used throughout the present specification refers to a straight or branched chain alkyl group having a carbon number of 1 to 4, which is a monovalent group derived by removing any hydrogen atom from a aliphatic hydrocarbon having a carbon number of 1 to 4, and as specific examples there may be mentioned methyl, ethyl, 1-propyl, 2-propyl and the like, with methyl being preferred.

The term “cyanophenyl” as used throughout the present specification refers to a phenyl group containing one cyano group, and specifically there may be mentioned 2-cyanophenyl, 3-cyanophenyl and 4-cyanophenyl, among which 3-cyanophenyl is preferred. The term “aminosulfonylphenyl” as used throughout the present specification refers to a phenyl group containing an aminosulfonyl group. The term “aminopyridyl” as used throughout the present specification refers to a pyridyl group containing an amino group. The term “aminopyrimidyl” as used throughout the present specification refers to a pyrimidyl group containing an amino group. The term “halogenopyridyl” as used throughout the present specification refers to a pyridyl group containing a halogen atom. The term “cyanothiophenyl” as used throughout the present specification refers to a thiophenyl group containing a cyano group.

A preparing process according to the invention will now be described. 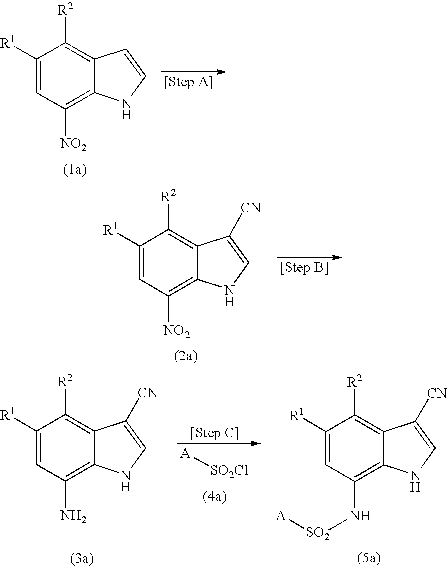
(In each formula, R¹, R² and A have the same definitions as above.)

(Step A)
This is a step of subjecting a compound (1a) to formylation reaction and then to cyanation reaction in the same reaction vessel without treatment of the reaction mixture, to afford a compound (2a).

A phosphorus oxyhalide or thionyl chloride is added to dimethylformamide in a temperature of −10° C. to 10° C., and the mixture is stirred at the same temperature for 10 minutes to 1 hour. A solution of a compound (1a) in dimethylformamide is then added at 0° C., and the mixture is heated and stirred at 10 to 60° C. for 30 minutes to 3 hours. This procedure results in formylation of compound (1a). Next, a solution of hydroxylamine hydrochloride in dimethylformamide is added to the reaction mixture while keeping the internal temperature below 80° C., and the mixture is heated and stirred at 10 to 60° C. for 30 minutes to 3 hours. Upon completion of the reaction, ordinary treatment, neutralization, extraction and purification are performed, if necessary, to afford a compound (2a).

The phosphorus oxyhalide may be phosphorus oxybromide or phosphorus oxychloride, and phosphorus oxychloride is preferred.

The phosphorus oxyhalide may be used at 1 to 3-fold as the molar ratio with respect to a compound (1a). The hydroxylamine may also be used at 1 to 3-fold as the molar ratio with respect to a compound (1a). A compound (1a) used as the starting material for this step may be synthesized by the preparing process described in WO00/50395.

The purification method employed may be purification by column chromatography using silica gel or an adsorption resin, or by recrystallization from an appropriate solvent.

(Step B)
This is a step of subjecting a compound (2a) to reduction reaction to afford a compound (3a). Any ordinary reduction reaction may be carried out for conversion of a nitro group to an amino group, but the reduction reaction is preferably catalytic reduction under a hydrogen atmosphere, in the presence of a catalytic reduction catalyst.

Specifically, a catalytic reduction catalyst is added to the reaction mixture containing a compound (2a), and reaction is carried out for 30 minutes to 24 hours under a hydrogen atmosphere at 1 to 5 atmospheres. Upon completion of the reaction, ordinary treatment, filtration, activated carbon treatment, extraction and purification are performed, if necessary, to afford a compound (3a).

The reaction solvent used may be a mixed solvent of tetrahydrofuran and methanol, or a mixed solvent of ethyl acetate and methanol, but preferably a mixed solvent of ethyl acetate and methanol (1:1) is used. The catalytic reduction catalyst used may be platinum oxide or 10% palladium-carbon, but preferably 10% palladium-carbon is used. The catalytic reduction catalyst may be used in an amount of 10 to 500-fold with respect to a compound (2a).

The purification method employed may be purification by column chromatography using silica gel or an adsorption resin, or by recrystallization from an appropriate solvent.

(Step C)

This is a step of reacting a compound (3a) with a compound (4a) to afford a compound (5a).

A compound (3a) and a compound (4a) are reacted at 20 to 80° C. in a mixed solvent of water and C₁₋₆ alkyl acetate, in the presence of a base. Upon completion of the reaction, ordinary treatment, neutralization, activated carbon treatment, extraction and purification are performed, if necessary, to afford a compound (5a).

A compound (4a) may be synthesized by the preparing process described in WO00/50395. The amount of a compound (4a) may be 0.8 to 1.3-fold with respect to a compound (3a), but it is preferably 1.1 -fold with respect to a compound (3a).

The reaction solvent used may be a mixed solvent of C₁₋₆ alkyl acetate and water in a volume ratio of 4:1 to 1:4, but preferably a mixed solvent of C₁₋₆ alkyl acetate and water in a volume ratio of 2:1 is used. Here, “C₁₋₆ alkyl acetate” means an ester compound where acetic acid bonds with C₁₋₆ alcohol, and specifically there may be mentioned methyl acetate and ethyl acetate, among which methyl acetate is preferred.

The base used may be pyridine, triethylamine, potassium carbonate, sodium hydrogencarbonate or the like. Pyridine may be mentioned as a preferred base. The base may be used at a molar ratio of 0.8 to 1.3 with respect to a compound (3a), but it is preferably used at a molar ratio of 1.2 with respect to a compound (3a).

### EXAMPLES

The present invention will now be explained in greater detail and specifically by the following examples, with the understanding that the invention is in no way limited to the examples.

### Example 1A

### Synthesis of 3-cyano-4-methyl-7-nitro-1H-indole

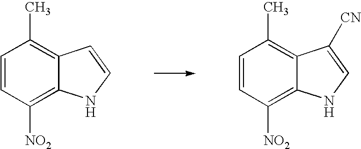

To 740 mL of dimethylformamide was added 235 mL (2.52 mol) of phosphorous oxychloride at 0° C., followed by stirring at 0° C. for 0.5 hour. To the reaction mixture was then added a solution of 370 g (2.10 mol) of 4-methyl-7-nitro-1H-indole (WO00/50395) in dimethylformamide (1110 mL) at 0° C., followed by heating and stirring at 60° C. for 2 hours.

To the reaction mixture was then added dropwise a solution of 292 g (4.20 mol) of hydroxylamine hydrochloride in dimethylformamide (1850 mL) with keeping the internal temperature below 80° C., followed by heating and stirring at 60° C. for 40 minutes. After adding 11.1 L of ice water to the reaction mixture while cooling in an ice bath, the mixture was further stirred overnight. The precipitated crystals were collected by filtration and washed with water. The crystals were suspended in 11.1 L of water, 1 N solution of sodium hydroxide was added to the suspension for adjustment to pH 7, and then the crystals were collected by filtration and washed with water to give 412 g of the title compound (yield: 97.6%).

HPLC analysis confirmed that the obtained compound was identical to the 3-cyano-4-methyl-7-nitro -1H-indole described in W00/50395.

(HPLC conditions)

Mobile phase: CH₃CN/H₂O/70% HClO₄=500/500/1 (v/v/v)
Flow rate: 1.0 mL/min
Detection: UV (254 nm)
Column: YMC-Pack Pro C18 250×4.6 mm

### Example 2A

### Synthesis of 7-amino-3-cyano-4-methyl-1H-indole

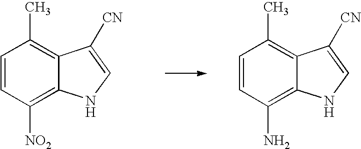

After suspending 400 g (1.99 mol) of the 3-cyano-4-methyl-7-nitro-1H-indole obtained in Example 1A in a mixture of 6 L of ethyl acetate and 6 L of methanol, the suspension was subjected to hydrogenation in the presence of 40 g of 10% palladium-carbon at ordinary temperature, 4 atmospheres. After removing the catalyst by filtration, the filtrate was treated with activated carbon and concentrated to give crude crystals. The crude crystals were dissolved in 6 L of 1,2-dimethoxyethane at an external temperature of 60° C., and then 12 L of water was added dropwise. Upon confirming precipitation of crystals, the mixture was stirred for 1.5 hours while cooling in an ice bath and filtered, and the crystals were washed twice with water (1 L). The crystals were air-dried at 50° C. for 16 hours to give 289 g of the title compound (yield: 84.8%).

HPLC analysis confirmed that the obtained compound was identical to the 7-amino-3-cyano-4-methyl-1H-indole described in WO00/50395.

(HPLC conditions)

Mobile phase: CH₃CN/H₂O/70% HClO₄=400/600/1 (v/v/v)
Flow rate: 1.0 mL/min
Detection: UV (282 nm)
Column: YMC-Pack Pro C18 250×4.6 mm

### Example 3A

### Synthesis of N-(3-cyano-4-methyl-1H-indol-7-yl)-3-cyanobenzenesulfonamide

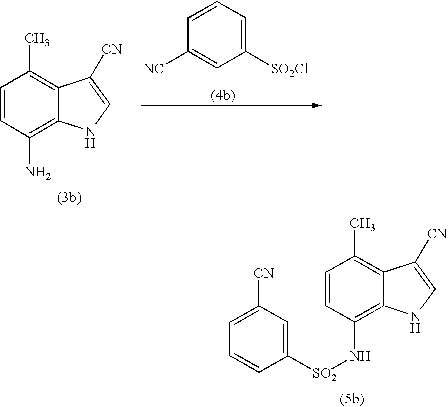

To a suspension of 5.0 g (29 mmol) of the 7-amino-3-cyano-4-methyl-1H-indole obtained in Example 2A and 6.48 g (32 mmol) of 3-cyanobenzenesulfonyl chloride [CAS No. 56542-67-7] in 150 mL of methyl acetate, were added 75 mL of water and 2.83 mL (35 mmol) of pyridine, followed by stirring for 2 hours and 40 minutes. After adding 0.73 mL (9 mmol) of concentrated hydrochloric acid to the reaction mixture, liquid-liquid separation was performed and the organic layer was washed with a mixture of 75 mL of water and 17.5 mL of ethanol. Activated carbon was added to the organic layer and the mixture was stirred at 45-50° C. for 30 minutes, and then filtered and concentrated. To thus obtained crude crystals were added 96 mL of 2-butanol and 24 mL of water for dissolution at 75° C., and the solution was cooled to 7° C at approximately 10° C./hr and stirred overnight. The precipitated crystals were collected by filtration and washed twice with 10 mL of 2-butanol to give 8.17 g (wet weight) of crystals of the title compound. The crystals were dried under reduced pressure at 70° C. for 2 hours to give 7.54 g of crystals of the title compound.

HPLC analysis confirmed that the obtained compound was identical to the N-(3-cyano-4-methyl-1H-indol-7-yl)-3-cyanobenzenesulfonamide described in WO00/50395.

(HPLC conditions)

Mobile phase: CH₃CN/H₂O/70% HClO₄=500/500/1 (v/v/v)
Flow rate: 1.0 mL/min
Detection: UV (282 nm)
Column: YMC-Pack Pro C18 250×4.6 mm

For comparison with Examples 1A-3A, Reference Examples 1A-3A were carried out based on the description of WO00/50359, and Reference Example 4A was carried out similarly to the description in WO00/50359.

### Reference Example 1A

### Synthesis of 3-formyl-4-methyl-7-nitro-1H-indole

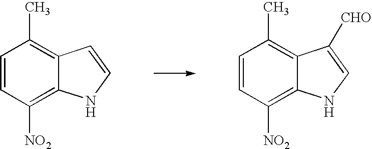

To 12 mL (154 mmol) of dimethylformamide was added 1.5 mL (16.1 mmol) of phosphorous oxychloride at 0° C., followed by stirring at the same temperature for 20.5 hour. To the reaction mixture was then added a solution of 2.0 g (11.4 mmol) of 4-methyl-7-nitro-1H-indole in dimethylformamide (20 mL) at 0° C., followed by heating and stirring at 90° C. for 21 hours. To the reaction mixture was added 100 mL of 1 N aqueous solution of sodium hydroxide to the reaction mixture while cooling in an ice bath, and extraction was performed with ethyl acetate. The organic layer was washed with water and brine in that order, dried over magnesium sulfate, and concentrated to dryness. A mixture of tert-butyl methyl ether and hexane was added to the residue, and the crystals were collected by filtration to give 2.23 g of the title compound (yield: 95.8%).

¹H-NMR (DMSO-d₆) δ (ppm): 2.90 (3H, s), 7.21 (1H, d, J=8.4Hz), 8.11 (1H, d, J=8.4Hz), 8.39 (1H, s), 10.01 (1H, s), 12.71 (1H, br s).

### Reference Example 2A

### Synthesis of 3-cyano-4-methyl-7-nitro-1H-indole

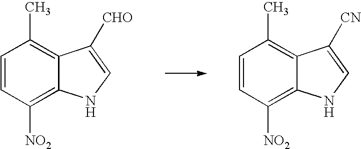

After dissolving 2.21 g (10.8 mmol) of the 3-formyl-4-methyl-7-nitro -1H-indole obtained in Reference Example 1A in 100 mL of dimethylformamide, 900 mg (13.0 mmol) of hydroxylamine hydrochloride and 1.05 mL (13.0 mmol) of pyridine were added. The mixture was heated and stirred at 60° C. for 40 minutes, and then 1,1′-carbonyldiimidazole (53.9 mmol) was added to the reaction mixture while cooling in an ice bath. The mixture was further heated and stirred at 60° C. for 30 minutes, and then 3.0 mL (21.5 mmol) of triethylamine was added to the reaction mixture, and heating and stirring were continued at the same temperature for 1 hour. To the reaction mixture was added 50 mL of ice water while cooling in an ice bath and extraction was performed with ethyl acetate. The organic layer was washed with water and brine in that order, dried over magnesium sulfate, and concentrated to dryness. A mixture of tert-butyl methyl ether and hexane was added to the residue, and the crystals were collected by filtration to give 1.95 g of the title compound (yield: 89.7%).

¹H-NMR (DMSO-d₆) δ (ppm): 2.78 (3H, s), 7.22 (1H, d, J=8.0Hz), 8.14 (1H, d, J=8.0Hz), 8.41 (1H, s), 12.76 (1H, br s).

### Reference Example 3A

### Synthesis of 7-amino-3-cyano-4-methyl-1H-indole

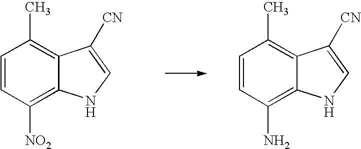

After suspending 12.6 g (62.6 mmol) of the 3-cyano-4-methyl-7-nitro-1H-indole obtained in Reference Example 2A in a mixture of 100 mL of tetrahydrofuran and 100 mL of methanol, the suspension was subjected to hydrogenation in the presence of 430 mg (1.87 mmol) of platinum oxide at ordinary temperature, 3 atmospheres. The catalyst was removed by filtration, the filtrate was concentrated to dryness, and then a mixture of tert-butyl methyl ether and hexane was added to the residue and the crystals were collected by filtration to give 10.7 g of the title compound (yield: 99.8%).

¹H-NMR (DMSO-d₆) δ (ppm): 2.47 (3H, s), 5.07 (2H, s), 6.34 (1H, d, J=7.6Hz), 6.64 (1H, d, J=7.6Hz), 8.10 (1H, s), 11.70 (1H, br s).

### Reference Example 4A

### Synthesis of N-(3-cyano-4-methyl-1H-indol-7-yl)-3-cyanobenzenesulfonamide

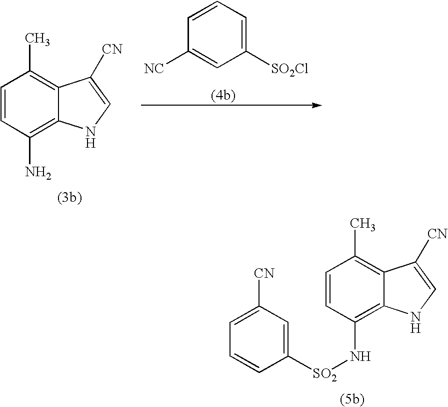

To a suspension of 250 g (1.46 mol) of the 7-amino-3-cyano-4-methyl-1H-indole obtained in Reference Example 3A in 5 L of tetrahydrofuran (20-fold amount), were added 354 mL (4.38 mol) of pyridine and 312 g (1.55 mol) of 3-cyanobenzenesulfonyl chloride, followed by stirring at an internal temperature of 21 to 34° C. Disappearance of the starting materials was confirmed after 30 minutes.

To the reaction mixture were added 2925 mL of water (11.7-fold amount), 5 L of ethyl acetate (20-fold amount) and a mixture of 730 mL of concentrated hydrochloric acid and 730 mL of water (total of 5.8-fold amount), and liquid-liquid separation was performed. The organic layer was washed with 2925 mL of water, and then 125 g of activated carbon was added and the mixture was stirred for 1 hour. The mixture was filtered through celite and washed twice with 1 L of ethyl acetate. To the filtrate were added 5 L of water and 100 mL of 1 N solution of sodium hydroxide, 1 L of ethyl acetate was further added and liquid-liquid separation was performed. Next, 6 L of water and 2 L of ethyl acetate were added to the organic layer and liquid-liquid separation was performed. The aqueous layer was re-extracted with 2 L of ethyl acetate, and both organic layers were combined and concentrated under reduced pressure at 50° C., after which 1 L of 2-propanol was added and azeotropic distillation and concentration was carried out to give the title compound (666 g, wet weight).

### Comparison of Example 1A and Reference Examples 1A and 2A

Reference Example 1A is a formylation step, and Reference Example 2A is a step of conversion from the formyl group to a cyano group. On the other hand, Example 1A accomplishes cyanation after formylation in the same reaction vessel, without reaction treatment such as extraction or solvent distillation (one-pot reaction).

As mentioned above, the yields in Reference Examples 1A and 2A are 95.8% and 89.7%, respectively, and the total yield of the two steps is 85.8%. In contrast, the yield in Example 1A is 97.6%. Thus, conducting the two reactions (formylation and cyanation) in one pot allowed the procedure to be simplified and the yield to be increased.

### Comparison of Example 3A and Reference Example 4A

The starting materials, reaction solvents, extraction solvents for the first addition after the reaction and the amounts of target compounds for Example 3A and Reference Example 4A are shown in Tables 1 and 2. The lowermost row in each table represents the amounts calculated per gram of the starting compound (3b).

TABLE 1

| Starting | | Reaction solvent | | | Extraction |
|---|---|---|---|---|---|
| material (g) | | (mL) | | | solvent (mL) |
| (3b) | (4b) | Methyl acetate | Water | Pyridine | Conc. HCl |
| 5.00 | 6.48 | 150 | 75 | 2.83 | 0.73 |
| 1.00 | 1.30 | 30 | 15 | 0.57 | 0.15 |

TABLE 2

| | | | | Extraction solvent | | |
|---|---|---|---|---|---|---|
| Starting | | Reaction | | (mL) | | |
| material (g) | | solvent (mL) | | Ethyl | Conc. HCl + | |
| (3b) | (4b) | THF | Pyridine | acetate | water | Water |
| 250 | 312 | 5000 | 354 | 5000 | 730 + 730 | 2925 |
| 1.00 | 1.25 | 20 | 1.42 | 20 | 2.92 + 2.92 | 11.7 |

The total volume of reaction solvent and extraction solvent required per gram of the starting compound (3b) was 58.96 mL in the process of Reference Example 4A, compared to 43.71 mL in the process of Example 3A.

Also, by the process of Reference Example 4A it is possible to carry out the reaction using approximately 16.96 g of compound (3b) per 1 L of reaction vessel for reaction and extraction, compared to approximately 22.88 g by the process of Example 3A. In other words, the process of Example 3A is more efficient as it permits more of the reaction to be carried out in the same reaction apparatus.

More specifically, the process of Example 3A allows the reaction to be accomplished with 1.4 times greater efficiency (5.92 g more per 1 L of reaction vessel) than the process of Comparative Example 4A.

### INDUSTRIAL APPLICABILITY

The process for preparing sulfonamide-containing indole compounds according to the invention has few reaction steps and a high yield, uses a small amount of solvent and is highly safe. It is therefore suitable as an industrial process for preparing sulfonamide-containing indole compounds useful as antitumor agents.

1. A process for preparing a compound (5a) represented by the following formula: 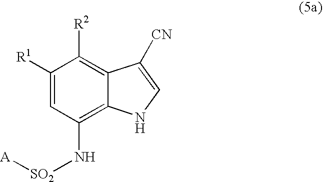
wherein R¹ and R² each independently represent hydrogen, C₁₋₄ alkyl or halogen, and A represents cyanophenyl, aminosulfonylphenyl, aminopyridyl, aminopyrimidyl, halogenopyridyl or cyanothiophenyl, characterized by reacting a compound (3a) represented by the following formula: 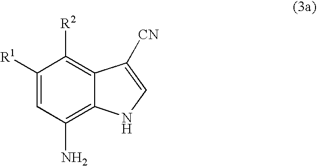
wherein R¹ and R² have the same definitions as above, with a compound represented by the formula A—SO₂Cl, wherein A has the same definition as above, in the presence of a base, in a mixed solvent of water and C₁₋₆ alkyl acetate.

2. A process for preparing a compound (5a) represented by the following formula: 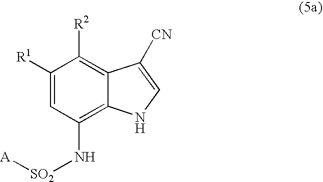
wherein R¹ and R² each independently represent hydrogen, C₁₋₄ alkyl or halogen, and A represents cyanophenyl, aminosulfonylphenyl, aminopyridyl, aminopyrimidyl, halogenopyridyl or cyanothiophenyl, characterized by reacting a compound (1 a) represented by the following formula: 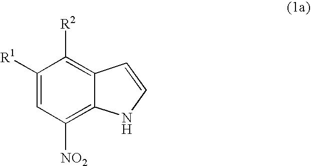
wherein R¹ and R² have the same definitions as above, with a phosphorus oxyhalide or thionyl chloride in dimethylformamide, then adding hydroxylamine hydrochloride to the reaction mixture to allow reaction therewith to afford a compound (2a) represented by the following formula: 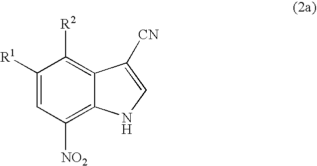
wherein R¹ and R² have the same definitions as above, and then subjecting the compound (2a) to reduction reaction to afford a compound (3a) represented by the following formula: 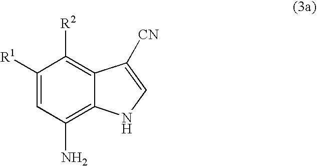
wherein R¹ and R² have the same definitions as above, and reacting the compound (3a) with a compound represented by the formula A—SO₂Cl, wherein A has the same definition as above, in the presence of a base, in a mixed solvent of water and C₁₋₆ alkyl acetate.

3. A process according to claim 1 or 2, wherein R² is methyl, R¹ is hydrogen and A is 3-cyanophenyl.
